(19) United States
(12) Patent Application Publication
(10) Pub. No.: US 2022/0062029 A1
(43) Pub. Date: Mar. 3, 2022

(54) FLUID COLLECTION DEVICES AND SYSTEMS

(71) Applicant: PureWick Corporation, El Cajon, CA (US)

(72) Inventors: Ashley Marie Johannes, Statham, GA (US); Martyn Mitchell, Hertfordshire (GB); Leanne Yip Heung Win, Middlesex (GB); Samuel Edmund Whittome, Cambridge (GB)

(21) Appl. No.: 17/446,654

(22) Filed: Sep. 1, 2021

Related U.S. Application Data
(60) Provisional application No. 63/074,066, filed on Sep. 3, 2020.

(51) Int. Cl. A61F 5/453; A61F 5/44

(52) U.S. Cl. CPC A61F 5/453; A61F 5/4401; A61F 5/4408

(57) ABSTRACT

A fluid collection device may include a fluid impermeable barrier and a fluid permeable body in some examples. In some examples, the fluid collection device may be primarily designed for use with female wearers but may be used with both male and female wearers with the use of an adapter and/or an attachment apparatus. In some examples, the fluid collection device may include shape memory materials that permit the fluid collection device to conform to a variety of anatomy types. In some examples, the fluid collection device may include customizable or predesigned contoured fluid permeable body to accommodate a variety of anatomy.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, can be used to address some of these circumstances. However, bed pans and urinary catheters have several problems associated therewith. For example, bed pans can be prone to discomfort, pressure ulcers spills, and other hygiene issues. Urinary catheters be can be uncomfortable, painful, and can cause urinary tract infections.

Recently, non-invasive fluid collection devices have been developed for use with immobile and/or limited mobility individuals. By non-invasive, it is meant that the catheter may be placed externally (e.g., outside an opening of the urethra). In some applications, non-invasive fluid collection devices may be designed for solely male or solely female wearers due to differences in external anatomy. However, the external anatomy of some wearers may not conform to typical male or female anatomy. For example, male wearers with excess adipose tissue may have “buried” anatomy that partially or completely obscures the shaft and/or head of the penis. In another example, some male wearers may have micro genitalia or suffer from retracted genitalia (e.g., due to side effects of certain medication). Fluid collection devices designed for typical male genitalia may be less effective or ineffective for these wearers.

Thus, users and manufacturers of fluid collection devices continue to seek new and improved devices and systems to collect bodily fluids that can accommodate a broader range of anatomy.

### SUMMARY

Embodiments disclosed herein are fluid collection devices, fluid collection systems, and components thereof.

In some embodiments, a fluid collection device may include a fluid impermeable barrier having an inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier, a fluid permeable body positioned at least partially within the chamber to extend across at least a portion of the opening and configured to wick fluid away from the opening, and a pouch defining a second opening and an interior, the interior surrounding a portion of the second end region, wherein the pouch is configured to accept at least a portion of a wearer's penis through the second opening into the interior.

In some embodiments a fluid collection device may include a fluid impermeable barrier having an inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier, a fluid permeable body positioned at least partially within the chamber to extend across at least a portion of the opening and configured to wick fluid away from the opening, and an attachment apparatus including an adhesive pad and a strap having a first end and a second end, wherein the first end is coupled to the adhesive pad and a second end is configured to be coupled to the fluid collection device.

In some embodiments, a fluid collection device may include a fluid impermeable barrier having an inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier, a fluid permeable body positioned at least partially within the chamber to extend across at least a portion of the opening and configured to wick fluid away from the opening, and an attachment apparatus including a belt, a fastener coupled to the belt, wherein the fastener is configured to secure a conduit coupled to a first end of the fluid collection device, and a strap having a first end coupled to the belt and a second end configured to be coupled to a second end of the fluid collection device opposite the first end of the fluid collection device.

In some embodiments, a fluid collection device may include a fluid impermeable layer having an inner surface at least partially defining a chamber, a first end region defining an aperture extending therethrough and including a first outer surface portion, and a second end region distal to the first end region, the fluid impermeable layer also defining an opening, a fluid permeable layer positioned at least partially within the chamber to extend across at least a portion of the opening and configured to wick fluid away from the opening, and a shape memory layer configured to maintain a shape into which the shape memory layer is manipulated into, wherein the fluid collection device defines at least one substantially planar surface that is manipulatable into a plurality of configurations.

In some embodiments, a fluid collection device may include a fluid impermeable barrier having an inner surface at least partially defining a chamber, wherein a bottom of the chamber is a substantially planar surface, the fluid impermeable barrier further defining a first end region defining an aperture extending therethrough and including a first outer surface portion, and a second end region distal to the first end region, the fluid impermeable barrier also defining an opening, a fluid permeable body positioned at least partially within the chamber to extend across at least a portion of the opening and configured to wick fluid away from the opening, and a retractable hood coupled to at least one of the fluid permeable body or the fluid impermeable membrane and configured to extend over at least a portion of the fluid permeable layer when in use, the retractable hood defining at least a portion of an opening and an interior, wherein the opening and interior are configured to accept at least a portion of a wearer's penis.

In some embodiments, a fluid collection device may include a fluid impermeable barrier having a concave inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier, wherein a perimeter of the opening defines an oblong shape, and a fluid permeable body disposed at least partially within the chamber including a plurality of blocks, wherein individual ones of the plurality of blocks are configured to be selectively removed to accommodate a wearer's anatomy.

In some embodiments, a fluid collection device may include a fluid impermeable barrier having a concave inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier, wherein a perimeter of the opening defines an oblong shape and a fluid permeable body at least partially disposed within the chamber including a plurality of contours to accommodate a wearer's anatomy.

In some embodiments, a fluid collection device may include a fluid impermeable barrier having a concave inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier, wherein a perimeter of the opening defines a teardrop shape, wherein a pointed end of the teardrop shape is at the first end region, a fluid permeable body positioned within at least a portion of the chamber to extend across at least a portion of the opening and configured to wick fluid away from the opening to a reservoir defined by the fluid permeable body and the fluid impermeable barrier, wherein the reservoir is disposed at the first end region, wherein a portion of the chamber occupied by the fluid permeable body is based, at least in part, on anatomy of a wearer, wherein the fluid collection device is configured to be worn by the wearer such that the first end region is gravitationally lower than the second end region.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric front view of a female fluid collection device according to at least one embodiment of the disclosure.

FIG. 1B is an exploded isometric view of the female fluid collection device of FIG. 1A.

FIG. 2 is a translucent view of an adapter provided with a fluid collection device according to at least one embodiment of the disclosure.

FIG. 3A is a front view of an attachment apparatus provided with a fluid collection device according to at least one embodiment of the disclosure.

FIG. 3B is a side view of the attachment apparatus and fluid collection device shown in FIG. 3A.

FIG. 4A is a front view of an attachment apparatus provided with a fluid collection device according to at least one embodiment of the disclosure.

FIG. 4B is a top-down view of a fastener of the attachment apparatus shown in FIG. 4A according to at least one embodiment of the disclosure.

FIG. 4C is a side view of the attachment apparatus and fluid collection device shown in FIG. 4A in a first position.

FIG. 4D is a side view of the attachment apparatus and fluid collection device shown in FIG. 4A in a second position.

FIG. 5A is a view of a fluid collection device in a first configuration according to at least one embodiment of the disclosure.

FIG. 5B is a view of the fluid collection device of FIG. 5A in a second configuration according to at least one embodiment of the disclosure.

FIG. 6A is a top-down view of a fluid collection device in a first configuration according to at least one embodiment of the disclosure.

FIG. 6B is an isometric view of the fluid collection device of FIG. 6A in the first configuration according to at least one embodiment of the disclosure.

FIG. 6C is an isometric view of the fluid collection device of FIG. 6A in a second configuration according to at least one embodiment of the disclosure.

FIG. 7A is an isometric view of an exterior of a fluid collection device according to at least one embodiment of the disclosure.

FIG. 7B is a front view of an interior of the fluid collection device of FIG. 7A.

FIG. 7C is a translucent side view of the fluid collection device FIG. 7A.

FIG. 8A is an isometric view of an exterior of a fluid collection device according to at least one embodiment of the disclosure.

FIG. 8B is a translucent side view of the fluid collection device of FIG. 8A.

FIG. 8C is a front view of an interior of the fluid collection device of FIG. 8A.

FIG. 8D is a front view of fluid permeable bodies which may be used to implement the fluid permeable body of FIG. 8A.

FIG. 9A is a front view of an interior of a fluid collection device according to at least one embodiment of the disclosure.

FIG. 9B is a cross-sectional view of the fluid collection device of FIG. 9A.

FIG. 10 is a schematic illustration of a system that includes a fluid collection device according to at least one embodiment of the disclosure.

### DETAILED DESCRIPTION

Embodiments disclosed herein are directed to fluid collection devices and portions thereof. The fluid collection devices disclosed herein are configured to collect fluids from an individual. The fluids collected by the fluid collection devices can include urine. The fluids collected by the fluid collection devices can also include at least one of vaginal discharge, penile discharge, reproductive fluids, blood, sweat, or other bodily fluids.

The fluid collection devices may include one or more components for drawing (e.g., wicking) fluid from a wearer and/or a surface of the fluid collection device into an interior of the fluid collection device. In some embodiments, the fluid collection devices may include a fluid permeable body for wicking fluid into the fluid collection device. In some embodiments, the fluid permeable body may include a fluid permeable membrane at least partially surrounding a support. In some embodiments, the support may be a fluid permeable support. In some embodiments, the fluid collection device may include a fluid permeable support without a fluid permeable membrane. The support alone or in combination with the fluid permeable membrane may direct fluids to an interior of the fluid collection device where the fluid may be removed from the fluid collection device.

As described herein, the fluid collection devices may be used on both male and female wearers and/or male or female wearers with non-standard anatomy (e.g., buried anatomy, micro anatomy).

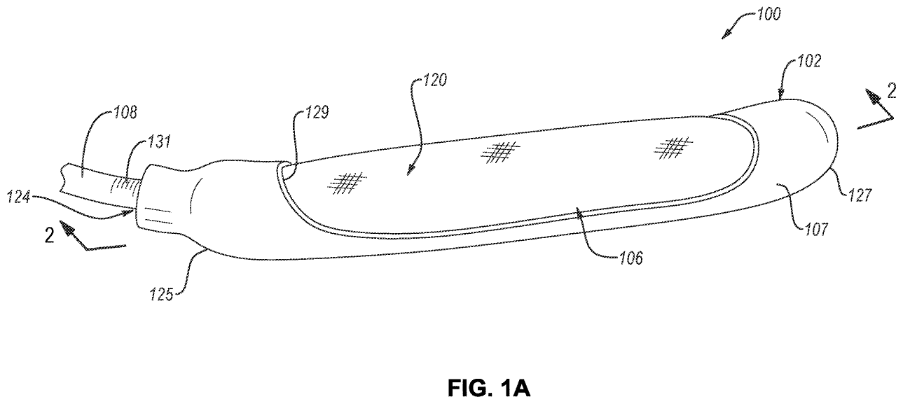

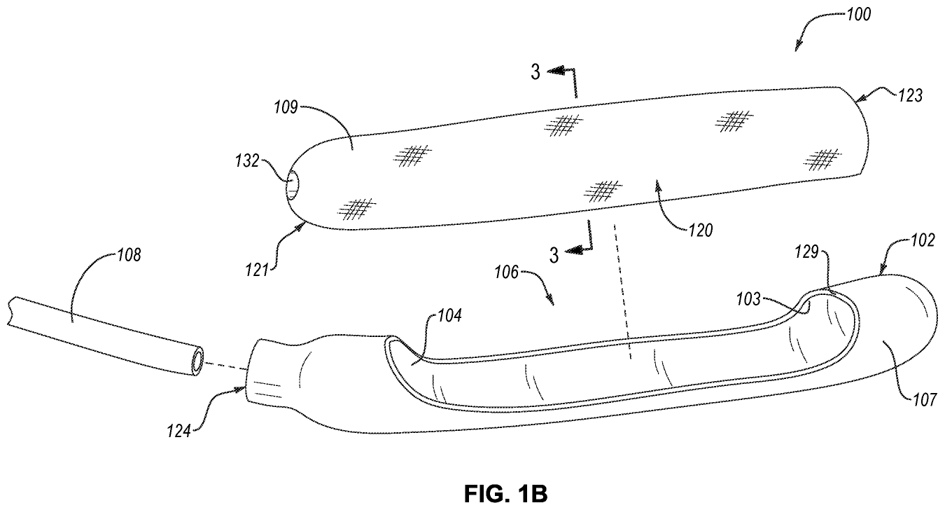

FIG. 1A is an isometric view of a fluid collection device 100, according to at least one embodiment of the disclosure, which may be used with one or more of the embodiments disclosed herein below. The fluid collection device 100 includes a fluid impermeable barrier 102 having a first end region 125 and a second end region 127. The first end region 125 may include an aperture 124 for accepting a conduit 108 that may couple the fluid collection device 100 to a vacuum source (not shown). The fluid impermeable barrier 102 at least partially defines a chamber 104 (e.g., interior region, shown in FIG. 1B) and includes an inward border or edge 129 defining an opening 106. The fluid impermeable barrier 102 may be substantially cylindrical in shape between the first end region 125 and the second end region 127. The opening 106 is formed in and extends through the fluid impermeable barrier 102, thereby enabling fluids to enter the chamber 104 from outside of the fluid collection device 100. The opening 106 can be configured to be positioned adjacent to a female urethra in some applications.

In some applications, the fluid collection device 100 may be positioned proximate to a urethra and urine may enter the interior region of the fluid collection device 100 via the opening 106. The fluid collection device 100 is configured to receive the fluids into the chamber 104 via the opening 106. For example, the opening 106 can exhibit an elongated shape that is configured to extend from a first location below the urethral opening (e.g., at or near the anus or perineum) to a second location above the urethral opening (e.g., at or near the pubic bone). The opening 106 can extend longitudinally along the fluid impermeable barrier. The opening 106 in the fluid impermeable barrier 102 can exhibit a width that is measured transverse to the longitudinal direction and may be at least about 10% of the circumference of the fluid collection device 100, such as about 25% to about 50%, about 40% to about 60%, about 50% to about 75%, about 65% to about 85%, or about 75% to about 100% of the circumference of the fluid collection device 100. The opening 106 can exhibit a width that is greater than 50% of the circumference of the fluid collection device 100 since a vacuum (e.g., suction) through a conduit 108 pulls the fluid into the conduit 108. In some embodiments, the opening 106 may be vertically oriented (e.g., having a major axis parallel to the longitudinal axis of the device 100). In some embodiments (not shown), the opening 106 may be horizontally oriented (e.g., having a major axis perpendicular to the longitudinal axis of the device 100). In some embodiments, the inward border or edge 129 of the fluid impermeable barrier 102 defines the opening 106. The edge 129 can include two opposing arced portions, the arcs following the outer circumference or periphery of the substantially cylindrical fluid impermeable barrier 102.

The fluid impermeable barrier 102 may also temporarily store the fluids in the chamber 104. As such, the fluid impermeable barrier 102 substantially prevents the fluids from exiting the portions of the chamber 104 that are spaced from the opening 106. The fluid impermeable barrier 102 may be flexible, allowing the fluid collection device 100 to bend or curve when positioned against the body of a wearer. For example, the fluid impermeable barrier 102 can be formed of any suitable fluid impermeable materials, such as a fluid impermeable polymer (e.g., silicone, polypropylene, polyethylene, polyethylene terephthalate, a polycarbonate, etc.), polyurethane films, TPE, oil, another suitable material, or combinations thereof. In some embodiments, the fluid impermeable barrier 102 can include one or more thermoplastic elastomers. The one or more thermoplastic elastomers may be combined with at least one of silicone and oil. In many embodiments, the fluid impermeable barrier 102 can include a composition having at least silicone and oil therein.

The fluid collection device 100 can include a fluid permeable body 120 or layer disposed in the chamber 104. The fluid permeable body 120 can cover or extend across at least a portion (e.g., all) of the opening 106. The fluid permeable body 120 can be configured to wick any fluid away from the opening 106, thereby preventing the fluid from escaping the chamber 104. The fluid permeable body 120 also can wick the fluid generally towards an interior of the chamber 104, as discussed in more detail below. A portion of the fluid permeable body 120 can define a portion of an outer surface of the fluid collection device 100. Specifically, the portion of the fluid permeable body 120 defining the portion of the outer surface of the fluid collection device 100 can be the portion of the fluid permeable body 120 exposed by the opening 106 defined by the fluid impermeable barrier 102. At least a portion of the fluid permeable body 120 exposed by the opening 106 may contact the wearer.

The fluid permeable body 120 can include any material that can wick the fluid. The permeable properties referred to herein can be wicking, capillary action, diffusion, or other similar properties or processes, and are referred to herein as “permeable” and/or “wicking.” Such “wicking” may exclude absorption into the wicking material (e.g., retention of fluid by the material). Put another way, substantially no absorption of fluid in the material may take place after the material is exposed to the fluid and removed from the fluid for a time. While no absorption is desired, the term “substantially no absorption” may allow for nominal amounts of absorption of fluid into the wicking material (e.g., absorbency), such as less than about 10 wt % of the dry weight of the wicking material, less than about 7 wt %, less than about 5 wt %, less than about 3 wt %, less than about 2 wt %, less than about 1 wt %, or less than about 0.5 wt % of the dry weight of the wicking material.

The fluid permeable body 120 can include a one-way fluid movement material. As such, the fluid permeable body 120 can remove fluid from the area around the urethra, thereby leaving the area dry. The fluid permeable body 120 can enable the fluid to flow generally into the chamber 104. For example, the fluid permeable body 120 can include a porous or fibrous material, such as hydrophilic polyolefin or spun nylon fibers. Examples of polyolefin that can be used in the fluid permeable body 120 include, but are not limited to, polyethylene, polypropylene, polyisobutylene, ethylene propylene rubber, ethylene propylene diene monomer, or combinations thereof. The porous or fibrous material can be extruded into a substantially cylindrically shape to fit within the chamber 104 of the fluid impermeable barrier 102. The fluid permeable body 120 can be manufactured according to various manufacturing methods, such as molding, extrusion, or sintering.

In some embodiments, during use, the fluid permeable body 120 extends from the conduit 108 to interface the fluid impermeable barrier 102 and the opening 106. In some embodiments, a majority of the outer surface 109 (shown in FIG. 1B) of the fluid permeable body 120 interfaces with an inner surface 103 (shown in FIG. 1B) of the fluid impermeable barrier 102. In other embodiments, a majority of the outer surface 109 may be exposed by the opening 106 of the fluid impermeable barrier 102. In some embodiments, at least a portion of the fluid permeable body 120 extends continuously across the opening 106 to wick any fluid from the opening 106 directly to the chamber 104. Moreover, when the fluid impermeable barrier is flexible and the fluid permeable body 120 is configured to wick fluid from the body rather than absorb fluid from the body and hold the fluid against the body, the fluid collection device 100, in some embodiments, is free from a seal or cushioning ring on the inward edge 129 defining the opening 106.

In some embodiments, the fluid permeable body 120 can include a fluid permeable membrane covering or wrapped around at least a portion of a fluid permeable support (not visible) with both the fluid permeable membrane and the fluid permeable support being disposed in the chamber 104. The fluid permeable membrane can cover or extend across at least a portion (e.g., all) of the opening 106. The fluid permeable membrane can be configured to wick any fluid away from the opening 106, thereby preventing the fluid from escaping the chamber 104. Fluids received in the chamber 104 of the fluid collection device 100 from the urethra can be removed through the conduit 108. The fluid permeable membrane may include a fabric such as gauze (e.g., a silk, linen, polymer based materials such as polyester or polypropylene, or cotton gauze), another soft fabric (e.g., jersey knit fabric or the like), or another smooth fabric (e.g., rayon, satin, or the like).

The conduit 108 may be at least partially disposed in the chamber 104. The conduit 108 (e.g., a tube) includes an inlet 110 at a second end region 127 of the fluid impermeable barrier 102 and an outlet 112 at a first end region 125 of the fluid impermeable barrier 102 positioned downstream from the inlet 110. The conduit 108 provides fluid communication between an interior region of the chamber 104 and a fluid storage container (not shown) or a portable vacuum source (not shown). For example, the conduit 108 may directly or indirectly fluidly couple the interior region of the chamber 104 and/or the reservoir 122 with the fluid storage container or the portable vacuum source.

In the illustrated embodiment, the fluid permeable body 120 defines a bore 132 extending through the fluid permeable body 120 from a first body end 121 of the fluid permeable body 120 to a second body end 123 of the fluid permeable body 120 distal to the first body end 121. In other embodiments, the bore 132 extends only partially into the fluid permeable body from the first body end 121 of the fluid permeable body 120.

In some embodiments, the conduit 108 is at least partially disposed in the chamber 104 and interfaces at least a portion of the bore 132 of the fluid permeable body 120. For example, the conduit 108 may extend into the fluid impermeable barrier 102 from the first end region 125 and may extend through the bore 132 to the second end region 127. The conduit 108 may extend beyond the fluid permeable body 120 or flush with the second body end 123. The fluid collected in the fluid collection device 100 may be removed from the interior region of the chamber 104 via the conduit 108. The conduit 108 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some embodiments, the conduit 108 may include silicone or latex.

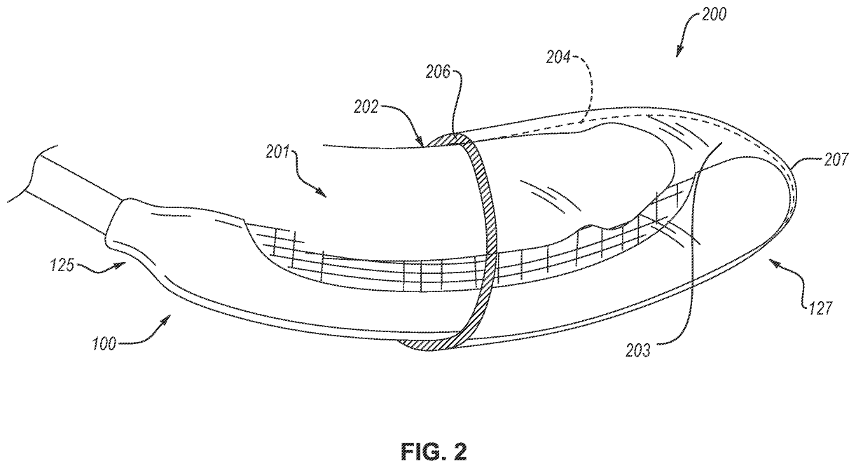

Additional details of the fluid collection device 100 may be found in U.S. application Ser. Nos. 15/171,968, 15/260,103, 15/611,587, 16/369,676, and 16/452,145, which are incorporated herein by reference for any purpose. The fluid collection device 100 shown in FIGS. 1A-B is typically used for collecting fluids from female wearers. However, as described herein, for example with reference to FIGS. 2-4, the fluid collection device 100 may also be used with male wearers with standard or nonstandard anatomy.

FIG. 2 is a translucent view of an adapter provided with a fluid collection device according to at least one embodiment of the disclosure. The adapter 200 may be a pouch defining an opening 202 into an interior 204 into which the second end region 127 of the fluid collection device 100 may be at least partially inserted. At least a portion of a penis 201 of a male wearer may also be inserted into the interior 204 of the adapter 200 between the fluid collection device 100 and the adapter 200. In some embodiments, the adapter 200 may be secured to the fluid collection device 100 and penis 201 by an elastic rim 206 at a perimeter of the opening 202. In some embodiments, the rim 206 may additionally or alternatively include an adhesive on at least a portion of an internal surface 203 at the rim 206. For example, an adhesive may be applied to the portion of the internal surface 203 adjacent to the fluid collection device 100.

In some embodiments, the adapter 200 may include a flexible fluid impermeable material. The adapter 200 may retain fluids from the penis 201 until the fluid is wicked away by the fluid collection device 100. In some embodiments, the adapter may include polyethylene, polypropylene, and/or polyvinyl chloride. In some embodiments, the adapter 200 may include multiple materials. For example, an external surface 207 may include a fluid impermeable material and the interior surface 203 may include a material that may or may not be fluid impermeable but may be selected based on providing comfort to the wearer's skin (e.g., microfiber fabric).

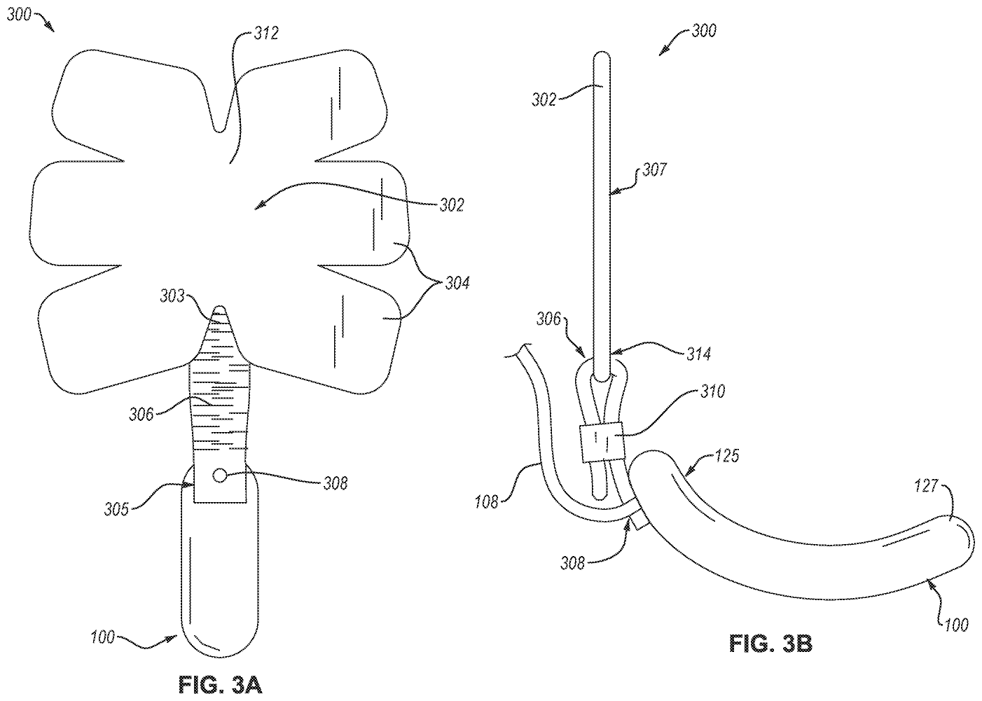

FIG. 3A is a front view of an attachment apparatus provided with a fluid collection device according to at least one embodiment of the disclosure. The attachment apparatus 300 may be used to help position and/or secure the fluid collection device 100 with respect to a female or male wearer in some applications. The attachment apparatus 300 may include an adhesive pad 302 and a strap 306.

In some embodiments, the adhesive pad 302 may include one or more tabs 304 extending from a central region 312 of the pad 302. The tabs 304 may facilitate movement by the wearer in some applications. In other embodiments, the tabs 304 may be omitted and the adhesive pad 302 may be substantially rectangular in shape. However, the adhesive pad 302 may define any desired shape (e.g., circular, triangular, elliptical). In some embodiments, the adhesive pad 302 may be sized to cover a majority of a wearer's abdomen (e.g., approximately from the pubic hair line to the sternum). In some embodiments, the adhesive pad 302 may be sized to cover a portion of the wearer's abdomen (e.g., approximately from the pubic hair line to the navel).

The strap 306 may be coupled to the adhesive pad 302 at a first end 303 and further coupled to the fluid collection device 100 at a second end 305. In some embodiments, the second end 305 may include an adhesive to couple the strap 306 to the fluid collection device 100. In some embodiments, the strap 306 may define a hole 308 through which the conduit 108 (shown in FIG. 3B) may pass. Alternatively, the strap 306 may be coupled adjacent to the conduit 108 or at an end of the fluid collection device 100 opposite the conduit 108 and the hole 308 may be omitted.

FIG. 3B is a side view of the attachment apparatus and fluid collection device shown in FIG. 3A. Optionally, in some embodiments, as shown in FIG. 3B, a length of the strap between the adhesive pad 302 and the fluid collection device 100 may be adjustable. In these embodiments, the strap 306 may pass through a hole 314 defined by adhesive pad 302. A fastener 310 may secure the strap 306 at a desired length. Any suitable fastening technique may be used to implement fastener 310. For example, fastener 310 may include a buckle, a strap, a clamp, a clip, hook-and-loop closures (e.g., Velcro), and/or one or more buttons. A user may pull a portion of the strap 306 through the hole 314 until the length between the adhesive pad 302 and the fluid collection device 100 is achieved. The user may then secure the fastener 310 to maintain the position.

In some embodiments, the adhesive pad 302 may include a flexible material with a low level adhesive on a surface 307 in contact with a user's skin during use. In some embodiments, the flexible material may include a woven and/or nonwoven material. In some embodiments, the flexible material may include a polymer such as PVC, polyethylene and/or polyurethane. In some embodiments, the flexible material may include a natural fiber such as linen and/or cotton. Examples of suitable adhesives may include, but are not limited to, a methacrylate and/or an epoxy diacrylate.

The attachment apparatus 300 may help secure at least one end region (e.g., first end region 125) of the fluid collection device 100 relative to a wearer. In some applications the other end of the fluid collection device 100 (e.g., second end region 127) may be secured, at least in part, by the wearer's legs and/or gluteal cleft. In some embodiments, the attachment apparatus 300 may be used in combination with the adapter 200.

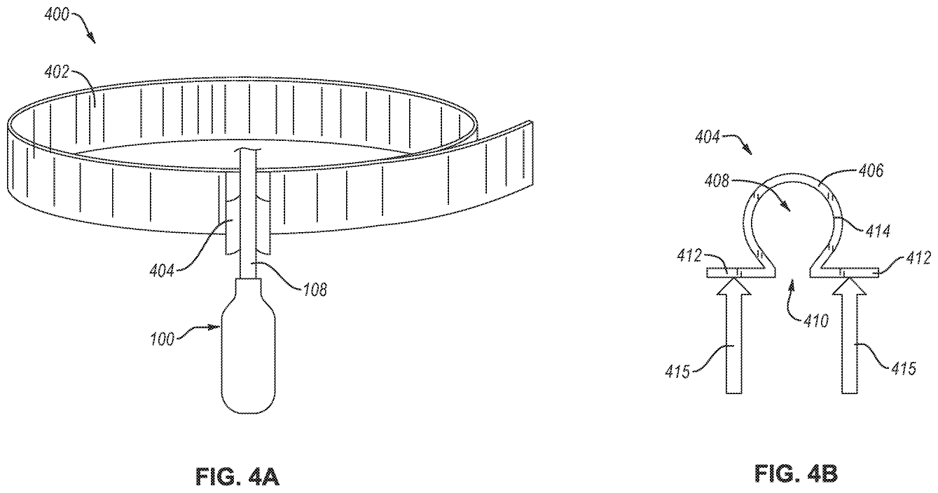

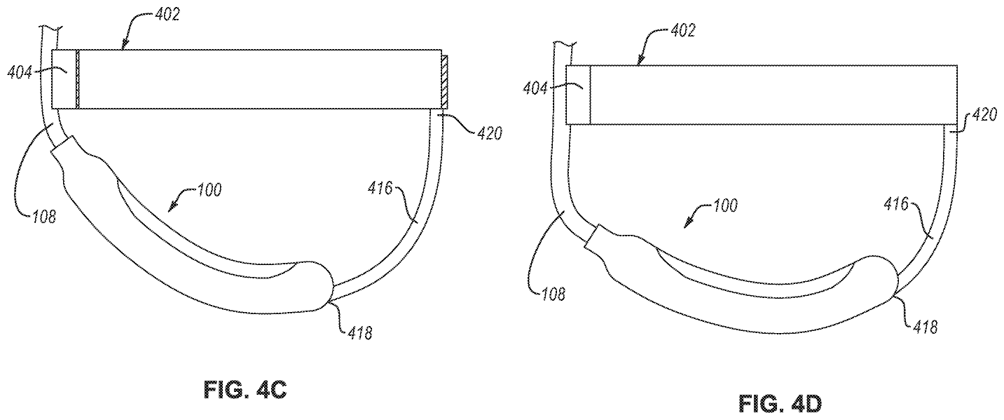

FIG. 4A is a front view of an attachment apparatus provided with a fluid collection device according to at least one embodiment of the disclosure. The attachment apparatus 400 may include a belt 402 and a fastener 404 coupled to the belt 402. The belt 402 may be worn around the hips or waist of a user of the fluid collection device 100. The fluid collection device 100 may be coupled to the attachment apparatus 400 via the conduit 108, which may be secured by the fastener 404. The belt 402 may include a woven or a nonwoven material in some embodiments. In some embodiments, the belt 402 may include nylon, polyester, cotton, and/or linen. In some embodiments, the belt 402 may be adjusted and secured at a desired width around the wearer by a buckle, hook-and-loop closures, buttons, and/or any other suitable securing technique (not shown).

FIG. 4B is a top-down view of a fastener of the attachment apparatus shown in FIG. 4A according to at least one embodiment of the disclosure. In some embodiments, the fastener 404 may include a clip as shown in FIG. 4B. The fastener 404 may include a rounded portion 406 that at least partially defines a channel 408 and an opening 410 into the channel 408. The fastener 404 may further include tabs 412 coupled to the rounded portion 406 and extending from either side of the opening 410.

The channel 408 may be sized to accept the conduit 108 and prevent slippage of the conduit 108 by pressure and/or friction. In some embodiments, an inner surface 414 of the channel 408 may be textured and/or include a nonslip coating to prevent movement of the conduit 108 within the channel.

The fastener 404 may be made of a resilient material that may permit temporary deformation. Example materials include polymers (e.g., nylon, polyacetal) and metals (e.g., steel, aluminum). In some embodiments, the opening 410 may be sized to be smaller than a diameter of the conduit 108 in resting state. However, the opening 410 may be temporarily widened by pressing the conduit 108 through the opening 410 and/or applying pressure to one or both tabs 412 as indicated by arrows 415. The conduit 108 may be inserted through the opening 410 into the channel 408 while the opening 410 is in its widened state. Once the conduit 108 is in the channel 408 and/or pressure on the tabs 412 is removed, the opening 410 may return to its resting state, thus securing the conduit 108 in the fastener 404.

Although the fastener 404 is shown as including a clip in FIG. 4B, in other embodiments, the fastener 404 may include other fastening implements. For example, the fastener 404 may include one or more adhesive strips, a zip-tie, and/or a clamp.

FIG. 4C is a side view of the attachment apparatus and fluid collection device shown in FIG. 4A in a first position. In some embodiments, the fluid collection device 100 may be further coupled to the belt 402 by a strap 416. A first end 418 of the strap 416 may be coupled to the fluid collection device 100. In some embodiments, the first end 418 may include an adhesive for coupling the strap 416 to the fluid collection device 100. A second end 420 of the strap 416 may be coupled to the belt 402. In some embodiments, the second end 420 may also include an adhesive to couple the strap 416 to the belt 402. In some embodiments, the second end 420 may be coupled to the belt 402 by stitches (not shown). Other coupling techniques may also be used.

In some embodiments, a length of the strap 416 may be adjustable. For example, the strap 416 may loop around the belt 402 and the attachment apparatus 400 may include a fastener such as a buckle (not shown) for securing the strap 416 at a desired length, similar to fastener 310 of attachment apparatus 300. In another example, the strap 416 may be cut to a desired length and/or include regularly spaced perforations for tearing off a portion of the strap 416 to achieve a desired length. In other embodiments, the length of the strap 416 may not be adjustable, but the “effective length” of the strap 416 may be changed based on where the first end 418 of the strap 416 is coupled to the fluid collection device 100. The adjustability of the strap 416 may permit the fluid collection device 100 to be secured in a variety of positions relative to the belt 402 and/or wearer.

For example, the fluid collection device 100 may be secured in a first position by the fastener 404 and strap 416. As shown in FIG. 4C, the first position may secure the fluid collection device 100 closer to an anterior of the wearer. FIG. 4D is a side view of the attachment apparatus and fluid collection device shown in FIG. 4A in a second position. In the second position, a length and/or effective length of the strap 416 is shorter than in FIG. 4C. The second position may secure the fluid collection device 100 closer to a posterior of the wearer.

Alternatively or additionally, the fluid collection device 100 may be secured in a variety of positions based on where along a length of the conduit 108, the fastener 404 is coupled to the conduit 108. Returning to FIG. 4C, the fastener 404 is coupled to the conduit 108 closer to the fluid collection device 100 than in FIG. 4D. Thus, a position of the fluid collection device 100 may be adjusted even if a length and/or effective length of the strap 416 is fixed. Although two positions are shown in FIGS. 4C-4D, it is understood that the fluid collection device 100 may be placed in any number of positions relative to the belt 402 and/or the wearer.

The attachment apparatus 400 may help secure both ends and/or an entirety of the fluid collection device relative to a wearer. In some embodiments, the attachment apparatus 400 may be used in combination with the adapter 200.

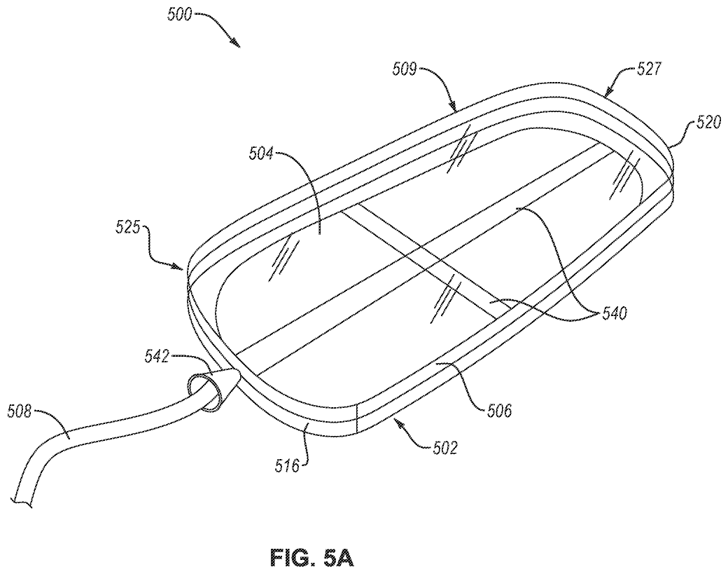

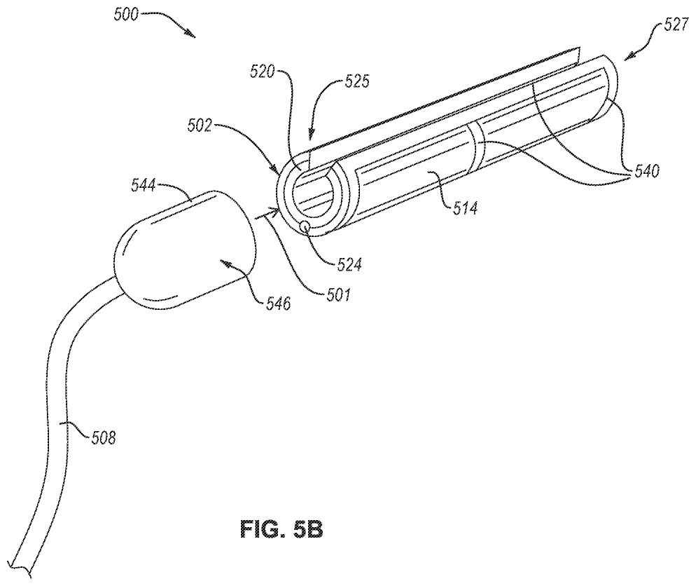

FIG. 5A is a view of a fluid collection device in a first configuration according to at least one embodiment of the disclosure. The fluid collection device 500 may include a fluid impermeable layer 502, a fluid permeable layer 520, and a shape memory layer 540. In the embodiment shown, the fluid collection device 500 has a substantially rectangular and planar shape, similar to a sanitary pad. However, other shapes may be provided for the fluid collection device 500 (e.g., round, square) depending on the application. In contrast to the substantially cylindrical shape of fluid collection device 100, the fluid collection device 500 may define a substantially flat, planar surfaces. However, as described in more detail, the fluid collection device 500 may be manipulated into a variety of configurations due, at least in part, to the shape memory layer 540.

In some embodiments, the fluid impermeable layer 502 includes a bottom surface 514 (indicated in FIG. 5B) and an edge 516 defining a chamber 504. The edge 516 may further define an opening 506 to the chamber 504. By bottom surface, it means a surface typically not in contact with a user's skin when the fluid collection device 500 is in use (e.g., worn by the user). The fluid impermeable layer 502 may define an aperture 524 (indicated in FIG. 5B) at a first end region 525 of the fluid collection device 500. In the embodiment shown, the aperture 524 is included in the edge 516. In some embodiments, the fluid impermeable layer 502 may include one or more materials that may be included in the fluid impermeable barrier of fluid collection device 100.

The fluid permeable layer 520 may be disposed adjacent to the fluid impermeable layer 502. In some embodiments, a portion of the fluid permeable layer 520 may be disposed within the chamber 504. The fluid permeable layer 520 may wick fluids from an outer surface of the fluid permeable layer 520 to the chamber 504. In some embodiments, the fluid permeable layer 520 may include one or more materials that may be included in fluid permeable body 120 of fluid collection device 100.

The shape memory layer 540 may include a material that may be manipulated and maintain the shape into which it was manipulated. Manipulation may include applying mechanical, thermal, and/or electrical forces to the shape memory layer 540. For example, a nurse may use their hands to bend the shape memory layer 540 as desired. In the embodiment shown, the shape memory layer 540 includes multiple strips. However, in other embodiments, the shape memory layer 540 may include a continuous layer, sheets, meshes, and/or a combination thereof. In some embodiments, the shape memory layer 540 may include metal, such as an elemental metal, an alloy, or shape memory alloy. Suitable shape memory metals may include standard steels, stainless steel, carbon alloy steel, head treated steel, aluminum, silver, copper, iron, nickel, zinc, tin, beryllium, or the like. Suitable shape memory alloys may include stainless steel; galvanized steel; aluminum alloys; nickel-titanium alloys, such as Nitinol, Ni—Ti—Cu, Ni—Ti, Co, or the like; copper-based alloys such as Cu—Zn—Al, Cu—Al—Ni, Cu—Al—Sn, or the like; Co—Cr—Ni—Mo alloys (e.g., Elgiloy®) or the like; or any other alloy having shape memory characteristics.

In some embodiments, the shape memory layer 540 may include a polymer. Shape memory polymers (“SMPs”) may include polyurethane-based SMPs such as a copolymer (e.g., copolyester, polyurethane, polyetherester, etc.) including blocks of one or more of poly(ε-caprolactone), polyethyleneterephthalate (PET), polyethyleneoxide (PEO), polyethylene glycol (PEG), polystyrene, polymethylmethacrylate (PMMA), Polybutylmethacrylate (PBMA), poly(N,N-butadiene), poly(N-methyl-N-oxazoline), polytetrahydrofuran, or poly(butylene terephthalate); thermoplastic polymers such as polyether ether ketone (PEEK), nylon, acetal, polytetrafluoroethylene (PTFE), polysulphone, or the like; Polynorbonene; or any other shape memory polymer.

In some embodiments, the shape memory layer 540 may be adjacent to the bottom surface 514 of the fluid impermeable layer 502. In some embodiments, the shape memory layer 540 may be disposed between the fluid impermeable layer 502 and the fluid permeable layer 520. In some embodiments, the shape memory layer 540 may be embedded within the fluid impermeable layer 502.

The shape memory layer 540 may permit the fluid collection device 500 to be shaped into a variety of configurations. In the example configuration shown in FIG. 5A, the fluid collection device 500 is substantially flat along a long axis and slightly curved along a short axis. In some applications, the curve along the short axis may encourage fluid to flow away from the edges of the fluid collection device 500. However, the fluid collection device 500 need not be curved along the short axis to be effective in all applications. Although not shown, the fluid collection device 500 could also be curved along the long axis, which may facilitate conforming the fluid collection device to female anatomy and/or buried male anatomy. In some configurations, such as the one shown in FIG. 5A, the fluid collection device 500 may look like and be used in a similar manner to a sanitary pad. In some embodiments, the bottom surface 514 the fluid impermeable layer 502 may include an adhesive, which may be used to secure the fluid collection device 500 to an undergarment of the wearer in the first configuration.

An end cap 542 may be inserted into the aperture 524 to couple a conduit 508 to the fluid collection device 500. The conduit 508 may remove fluids from the chamber 504 via the aperture 524. In some embodiments, the conduit 508 may be substantially the same as conduit 108 of fluid collection device 100.

FIG. 5B is a view of the fluid collection device of FIG. 5A in a second configuration according to at least one embodiment of the disclosure. In the example configuration shown, the fluid collection device 500 is substantially flat along the long axis and curved along the short axis to form a substantially tubular shape. Although shown as having a gap in the tube, in some configurations, the edges of the fluid collection device 500 may be flush with one another forming a continuous tube. A portion of the fluid collection device 500 proximate to the first end region 525 may be inserted into an end cap 544 as indicated by arrow 501. In contrast to end cap 542, which was inserted into the aperture 524, the opening 546 of the end cap 544 may encompass the entire “tube” of the fluid collection device in some embodiments. The end cap 544 may fluidly couple the conduit 508 to the aperture 524. In some embodiments, the conduit 508 may extend through the end cap 544 and be inserted into the aperture 524.

In some applications, when the fluid collection device 500 is in the configuration shown in FIG. 5B, a portion of a penis of a wearer may be inserted into the “tube” from the second end region 527 of the fluid collection device. In some applications, rather than inserting the wearer's penis into the tube, the fluid collection device 500 may be manipulated to form a tubular shape around the wearer's penis. Thus, in some applications, the fluid collection device 500 may be suitable for female wearers as well as male wearers with standard and non-standard anatomy.

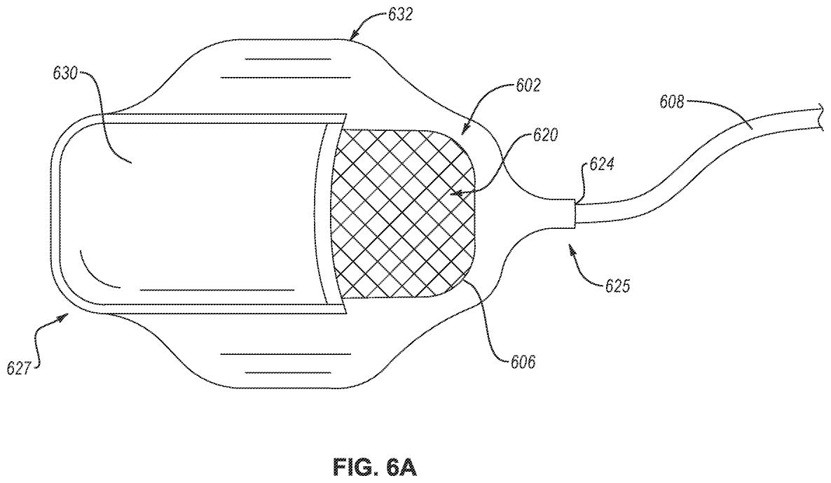

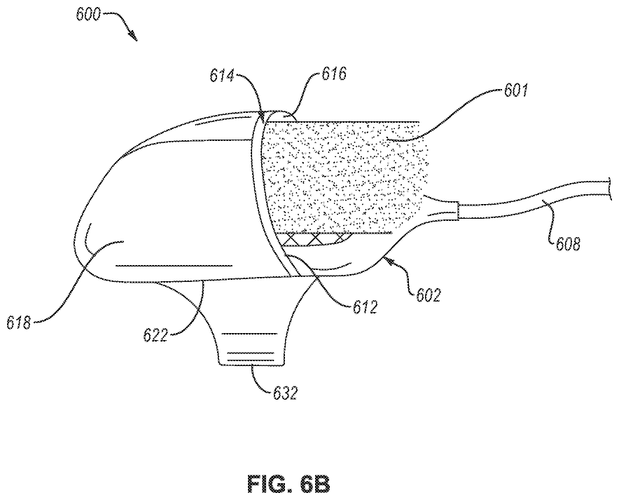

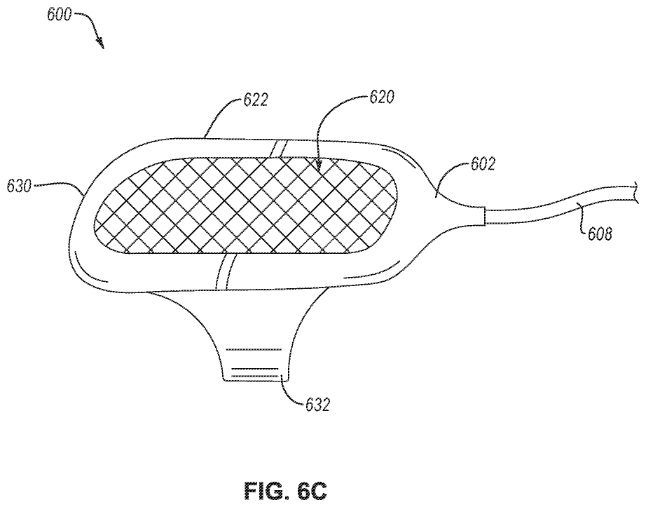

FIG. 6A is a top-down view of a fluid collection device in a first configuration according to at least one embodiment of the disclosure. By “top” it is meant at least some of the surfaces of the fluid collection device 600 typically in contact with a wearer's skin when in use are fully or partially visible. In some embodiments, the fluid collection device 600 may have a shape and arrangement similar to a winged sanitary pad. The fluid collection device 600 may include a fluid impermeable barrier 602, a fluid permeable body 620 disposed adjacent to the fluid impermeable barrier 602, and a retractable hood 630. In the configuration shown in FIGS. 6A-B, the hood 630 is extended over at least a portion of the fluid permeable body 620.

The fluid impermeable barrier 602 may at least partially define an opening 606 into a chamber (not shown), similar to fluid impermeable barrier 102, albeit a different shape. Similar to fluid impermeable layer 502, a bottom surface of the chamber may define a substantially planar surface, rather than a curved or cylindrical surface as chamber 104 of fluid collection device 100. The fluid impermeable barrier 602 may further define an aperture 624 at a first end region 625 of the fluid collection device 600. In some embodiments, the fluid impermeable barrier 602 may include one or more materials that may be included in fluid impermeable barrier of fluid collection device 100. A conduit 608 may be coupled to and/or pass through the aperture 624. In some embodiments, the conduit 608 may extend at least partially through the chamber of the fluid impermeable barrier 602. The conduit 608 may be fluidly connected to the chamber to remove fluid from the fluid collection device 600. In some embodiments, the conduit 608 may be substantially the same as conduit 108.

The fluid permeable body 620 may be disposed adjacent to the fluid impermeable barrier 602. In some embodiments, a portion of the fluid permeable body 620 may be disposed within the chamber of the fluid impermeable barrier 602. The fluid permeable body 620 may wick fluids from an outer surface of the fluid permeable body 620 to the chamber. In some embodiments, the fluid permeable body 620 may include one or more materials that may be included in fluid permeable body 120 of fluid collection device 100.

In some embodiments, such as the one shown, the fluid impermeable barrier 602 may extend on either side of the fluid permeable body 620 to form wings 632. In some embodiments, the fluid impermeable barrier 602 may include an adhesive on a bottom surface (not shown) opposite a surface adjacent to the fluid permeable body 620. In some applications, the adhesive may be used to secure the fluid collection device 600 to an undergarment of a wearer. In some embodiments, the wings 632 may further secure the fluid collection device 600 to the undergarment. For example, the wings 632 may adhere to the inner surface of leg portions of the undergarment. In another example, the fluid collection device 600 may be secured to an upper surface of a crotch portion of the undergarment and the wings 632 may be folded and secured to a lower surface of the crotch portion.

FIG. 6B is an isometric view of the fluid collection device 600 in the first configuration according to at least one embodiment of the disclosure. As noted, the hood 630 may extend over at least a portion of the fluid permeable body 620 in the first configuration. The first configuration may arrange the fluid collection device 600 for use by a male wearer. In the first configuration, the hood 630 may define at least a portion of an opening 612 to an interior 616. At least a portion of a wearer's penis 601 may be inserted through the opening 612 and into the interior 616 between the fluid collection device 600 and the hood 630. In some embodiments, the hood 630 may include an elastic strip 614 about a perimeter of the opening 612 to retain the penis 601 in the interior 616. In some embodiments, an inner edge of the opening 612 may include an adhesive to retain the penis 601 in the interior 616.

In some embodiments, the hood 630 may include a flexible fluid impermeable material. The hood 630 may retain fluids from the penis 601 until the fluid is wicked away by the fluid permeable body 620. In some embodiments, the hood 630 may include polyethylene, polypropylene, and/or polyvinyl chloride. In some embodiments, the hood 630 may include multiple materials. For example, an external surface 618 may include a fluid impermeable material and the inner surface of the interior 616 may include a material that may or may not be fluid impermeable but may be selected based on providing comfort to the wearer's skin (e.g., microfiber fabric).

In some embodiments, an edge 622 of the hood 630 may be coupled to the fluid impermeable barrier 602. In some embodiments, the edge 622 may be coupled to the fluid permeable body 620. The edge 622 may be coupled to the fluid impermeable barrier 602 and/or fluid permeable body 620 by stitching, ultrasonic welding, an adhesive, and/or other suitable coupling technique.

FIG. 6C is an isometric view of the fluid collection device 600 in a second configuration according to at least one embodiment of the disclosure. In the second configuration, the hood 630 may be folded under the fluid permeable body 620. In these embodiments, the fluid permeable body 620 may be at least partially removable from the fluid impermeable barrier 602 to permit the hood 630 to be inserted between the fluid permeable body 620 and the fluid impermeable barrier 602. Additionally or alternatively, the hood 630 may be removable such that the hood 630 may be detached from the fluid collection device 600 if not used. In some embodiments, the hood 630 may not be necessary when fluid collection device 600 is used by a female wearer and/or a male wearer with buried anatomy. Thus, in some applications, the fluid collection device 600 may be suitable for female wearers as well as male wearers with standard and non-standard anatomy.

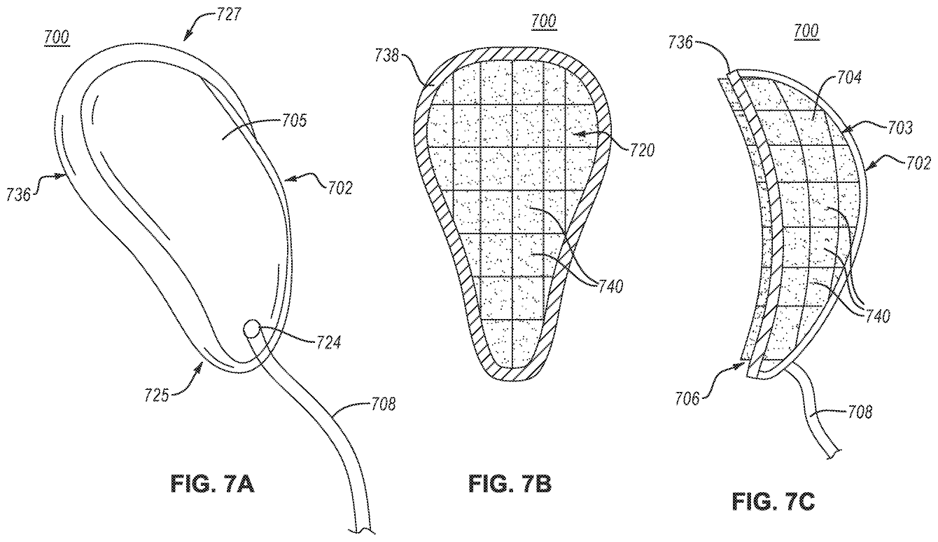

FIG. 7A is an isometric view of an exterior of a fluid collection device according to at least one embodiment of the disclosure. In some embodiments, the fluid collection device 700 may include a fluid impermeable barrier 702 and a fluid permeable body 720.

In some embodiments, the fluid impermeable barrier 702 may include silicone, closed cell foam, poly(p-phenylene ether), and/or any other materials described as included in fluid impermeable barrier 102 of FIG. 1. The fluid impermeable barrier 702 may at least partially define a chamber 704 and an opening 706 to the chamber 704 (indicated in FIG. 7C). Similar to the fluid impermeable barrier 102, the fluid impermeable barrier 702 may accept fluid into the chamber 704 through opening 706. The fluid impermeable barrier 702 may further define an aperture 724 at a first end 725. The aperture 724 may pass through the fluid impermeable barrier 702 into the chamber 704. The aperture 724 may be sized to accept a conduit 708. Similar to conduit 108, the conduit 708 may receive fluids from the chamber 704 and remove the fluids from the chamber 704. In some embodiments, the conduit 708 may extend at least partially into the chamber 704. In some embodiments, the conduit 708 may extend through the chamber 704 at or near a second end 727 of the fluid impermeable barrier 702.

In contrast to the generally cylindrical fluid impermeable barrier 102, the fluid impermeable barrier 702 may have a curved ovular shape and/or rounded “teardrop” shape, or more generally an oblong shape, similar to a protective athletic cup. An inner surface 703 (indicated in FIG. 7C) of the fluid impermeable barrier 702 may be generally concave whereas an outer surface 705 may be generally convex. The fluid impermeable barrier 702 may generally be shaped to curve around a wearer's crotch and/or pubic area. For example, in some embodiments, the first end 725 may be proximate the wearer's pubic bone whereas the second end 727 is below a user's urethra (e.g., proximate the user's perineum). In some embodiments, such as when the conduit 708 does not extend into the chamber 704, the second end 727 may be proximate the wearer's pubic bone whereas the first end 725 may be proximate the wearer's perineum. In this configuration, the first end 725 may be lower than the second end 727, which may facilitate removal of fluid from the chamber 704.

In some embodiments, the fluid collection device 700 may further include a liner 736 at a perimeter of the opening 706. The liner 736 may allow for air flow and/or prevent leaks. The liner 736 may further provide a comfortable fit and/or feeling against the skin of the wearer. The liner 736 may include an open cell foam in some embodiments. In some embodiments, the liner 736 may include a hydrophobic material and/or a hydrophobic coating, which may reduce or prevent leaks of fluid from the opening 706. In some embodiments, the liner 736 may include an adhesive on an edge 738 (indicated in FIG. 7B) adjacent to the wearer's skin when in use to maintain the position of the fluid collection device 700 on the wearer. In other embodiments, the fluid collection device 700 may be held in place by mesh pants (not shown) or other undergarment worn by the wearer over the fluid collection device 700.

FIG. 7B is a front view of an interior of the fluid collection device 700. As shown, in some embodiments, a perimeter of the opening 706 may define a rounded teardrop or pear-like shape. The fluid collection device 700 may include a fluid permeable body 720. Similar to fluid permeable body 120, the fluid permeable body 720 may wick fluids away from the wearer and facilitate fluid flow through the chamber 704 to the conduit 708 for removal from the fluid collection device 700. The fluid permeable body 720 may be at least partially disposed within the chamber 704 of the fluid impermeable barrier 702 and be at least partially exposed by the opening 706. In the embodiment shown, the fluid permeable body 720 may include multiple blocks 740 of fluid wicking material. In some embodiments, the blocks 740 may be formed by scoring and/or partial scoring a fluid wicking material. In some embodiments, the blocks 740 may include an open cell foam such as open cell polyurethane foam.

FIG. 7C is a translucent side view of the fluid collection device 700. In some embodiments, the fluid permeable body 720 may be coupled to the inner surface 703 of the fluid impermeable barrier 702 to retain the fluid permeable body 720. In some embodiments, the fluid permeable body 720 may be retained by a wearer's skin when the fluid collection device 700 is worn. In some embodiments, the fluid permeable body 720 may extend beyond the liner 736. In some applications, individual blocks 740 of the fluid permeable body 720 may be selectively removed (e.g., “pick-and-pluck” foam) to provide a customized fit for a wearer's anatomy. For example, in some applications, no blocks 740 may be removed for female wearers and/or male wearers with buried anatomy, and the fluid collection device 700 may be placed adjacent to the wearer's urethral opening. In other applications, some blocks 740 on either side of a central region of the chamber 704 may be removed such that a ridge of blocks 740 may be placed between a female wearer's labia. In some applications, any number and arrangement of blocks 740 may be removed to accommodate various sizes and shapes of male wearers' anatomy. Accordingly, the fluid collection device 700 may provide a customizable fit for a variety of anatomy for both female and male wearers.

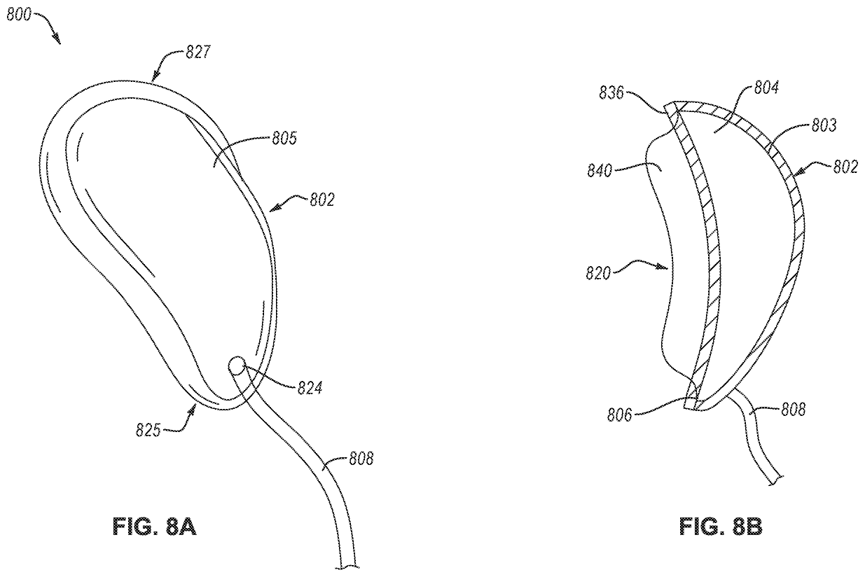

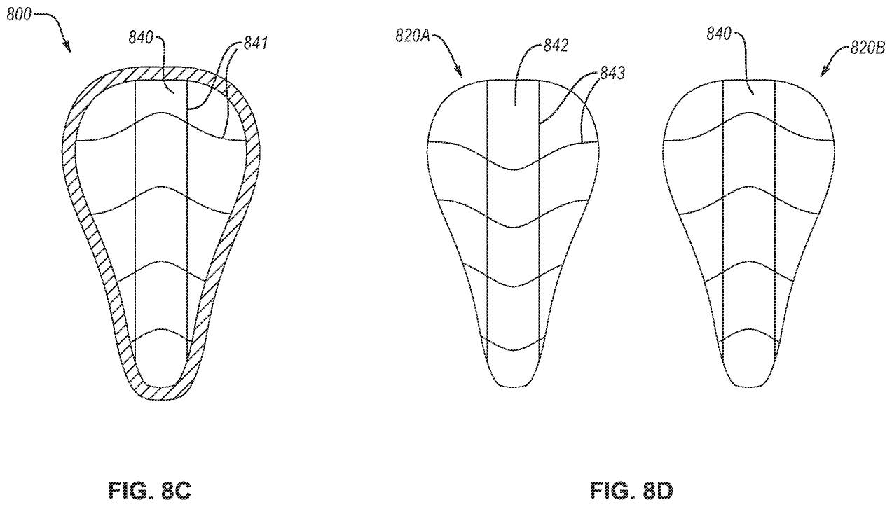

FIG. 8A is an isometric view of an exterior of a fluid collection device according to at least one embodiment of the disclosure. The fluid collection device 800 may include a fluid impermeable barrier 802, a liner 836, and a conduit 808. In some embodiments, these components of fluid collection device 800 may be substantially the same as fluid impermeable barrier 702, liner 736, and conduit 708 shown in FIGS. 7A-C. Accordingly, for brevity, these components will not be described in detail.

FIG. 8B is a translucent side view of the fluid collection device 800. The fluid collection device 800 may include a fluid permeable body 820. Similar to fluid permeable body 120, the fluid permeable body 820 may wick fluids away from the wearer and facilitate fluid flow through the chamber 804 to the conduit 808 for removal from the fluid collection device 800. The fluid permeable body 820 may be at least partially disposed within the chamber 804 of the fluid impermeable barrier 802 and be at least partially exposed by the opening 806. As shown, in some embodiments, the fluid permeable body 820 may extend beyond liner 836. In some embodiments, the fluid permeable body 820 may be coupled to the inner surface 803 of the fluid impermeable barrier 802 to retain the fluid permeable body 820. In some embodiments, the fluid permeable body 820 may be retained by a wearer's skin when the fluid collection device 800 is worn.

In some embodiments, the fluid permeable body 820 includes an open cell foam, such as a polyurethane open cell foam. In some embodiments, the fluid permeable body 820 may include one or more materials included in fluid permeable body 120. Although not shown, in some embodiments, the fluid permeable body 820 may include a support at least partially covered by a fluid permeable membrane.

FIG. 8C is a front view of an interior of the fluid collection device 800. In contrast to the fluid permeable body 720 of fluid collection device 700, the fluid permeable body 820 may be pre-molded to accommodate a wearer's anatomy. For example, the fluid permeable body 820 may be pre-molded with various contours as indicated by lines 841. In the example shown in FIGS. 8B and 8C, the fluid permeable body 820 is molded with a protrusion 840 for inserting between the labia of a female wearer. However, other contours may also be provided.

FIG. 8D is a front view of fluid permeable bodies 820A, 820B, which may be used to implement the fluid permeable body 820. The fluid permeable body 820B includes the same contours as those shown in the example of FIGS. 8B and 8C. As indicated by lines 843, the fluid permeable body 820A is contoured with a depression 842 rather than a protrusion, such as protrusion 840. The depression 842 may accommodate male anatomy (e.g., at least a portion of the penis and/or scrotum). The contours of fluid permeable bodies 820A, 820B are provided as examples, and the fluid permeable body 820 is not limited to the examples shown, and the fluid permeable body 820 may be contoured in any suitable manner to accommodate a wearer's anatomy, whether male or female with standard or nonstandard anatomy.

In some embodiments, the fluid collection device 800 may come with a fluid permeable body 820 with the desired contours already disposed within the chamber 804. In some embodiments, the fluid collection device 800 may come as a kit with multiple fluid permeable bodies 820 that may be selected by a user and inserted into the chamber 804. In some embodiments, the fluid permeable body 820 may be custom contoured for a particular wearer.

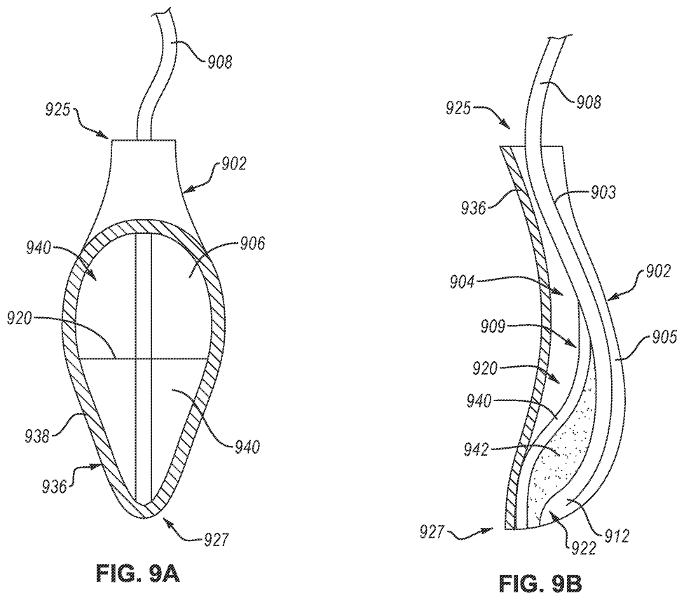

FIG. 9A is a front view of an interior of a fluid collection device according to at least one embodiment of the disclosure. The fluid collection device 900 may include a fluid impermeable barrier 902 and a fluid permeable body 920.

In some embodiments, the fluid impermeable barrier 902 may include silicone, closed cell foam, poly(p-phenylene ether), and/or any other materials described as included in fluid impermeable barrier 102 of FIG. 1. The fluid impermeable barrier 902 may at least partially define a chamber 904 and an opening 906 to the chamber 904. Similar to the fluid impermeable barrier 102, the fluid impermeable barrier 902 may accept fluid into the chamber 904 through opening 906. The fluid impermeable barrier 902 may further define an aperture 924 at a first end 925. The aperture 924 may pass through the fluid impermeable barrier 902 into the chamber 904. The aperture 924 may be sized to accept a conduit 908.

Somewhat similar to the fluid impermeable barriers 702 and 802, may have a curved ovular shape and/or “teardrop” shape. However, as will be discussed in more detail, the fluid impermeable barrier 902 may have a more “pointed” teardrop shape at the second end 927 in comparison to the fluid impermeable barriers 702 and 802. An inner surface 903 (indicated in FIG. 9B) of the fluid impermeable barrier 902 may be generally concave whereas an outer surface 905 may be generally convex. The fluid impermeable barrier 902 may generally be shaped to curve around a wearer's crotch and/or pubic area.

In some embodiments, the fluid collection device 700 may further include a liner 936 at a perimeter of the opening 906. The liner 936 may allow for air flow and/or prevent leaks. The liner 936 may further provide a comfortable fit and/or feeling against the skin of the wearer. The liner 936 may include an open cell foam in some embodiments. In some embodiments, the liner 936 may include a hydrophobic material and/or a hydrophobic coating, which may reduce or prevent leaks of fluid from the opening 906. In some embodiments, the liner 936 may include an adhesive on an edge 938 adjacent to the wearer's skin when in use to maintain the position of the fluid collection device 900 on the wearer. In other embodiments, the fluid collection device 900 may be held in place by mesh pants (not shown) or other undergarment worn by the wearer over the fluid collection device 900.

FIG. 9B is a cross-sectional view of the fluid collection device 900. As noted above, the aperture 924 may be sized to accept conduit 908. Similar to conduit 108, the conduit 908 may receive fluids from the chamber 904 and remove the fluids from the chamber 904. In some embodiments, the conduit 908 may provide suction to the chamber 904 (e.g., via a vacuum source) to facilitate removal of fluid from the fluid collection device 900. As is shown, conduit 908 may extend to a second end 927 of the fluid collection device 900 to a reservoir 922. The reservoir 922 may be at least partially define by the inner surface 903 of the fluid impermeable barrier 902. The reservoir 922, may be further defined, at least in part, by the fluid permeable body 920. Similar to fluid permeable body 120, the fluid permeable body 920 may wick fluids away from the wearer and facilitate fluid flow through the chamber 904 to the reservoir 922. In some embodiments, the reservoir 922 may be a substantially empty volume of the fluid collection device 900. In some embodiments, the fluid collection device 900 may be worn such that the second end 927 is gravitationally lower than the first end 925, which may facilitate fluid flow to the reservoir 922. Fluid may temporarily be stored in the reservoir 922 prior to being removed via inlet 912 of the conduit 908. The narrow shape of the fluid impermeable barrier 902 at the second end 927 may facilitate gathering the fluid into a smaller volume which may improve removal of the fluid from the reservoir 922 by the conduit 908.

The fluid permeable body 920 may include a fluid permeable support 942 at least partially covered by a fluid permeable membrane 940. In some embodiments, the fluid permeable support 942 may include an open cell foam. In some embodiments, the fluid permeable membrane 940 may include a nonwoven material, such as a polyethylene top sheet, selected to provide comfort (e.g., soft, non-chafing) for a wearer's skin. In some embodiments, the fluid permeable body 920 may include one or more materials included in fluid permeable body 120. In some embodiments, the fluid permeable body 920 may be coupled to the inner surface 903 of the fluid impermeable barrier 802 to retain the fluid permeable body 920. In some embodiments, the fluid permeable body 920 may be retained by a wearer's skin when the fluid collection device 900 is worn.

As shown in FIGS. 9A-B, in some embodiments, the fluid permeable body 920 may be entirely within the chamber 904 and only partially fill the chamber 904. In some embodiments, an outer surface 909 of the fluid permeable body 920 may be contoured to accommodate a wearer's anatomy. Thus, in some applications, a portion of the wearer's anatomy may be at least partially enclosed by the chamber 904. In some embodiments, shape of the contour of the outer surface 909 and/or an amount of the chamber 904 occupied by the fluid permeable body 920 may vary depending on the intended wearer of the fluid collection device. For example, less of the chamber 904 may be occupied by the fluid permeable body 920 when the intended wearer is a male with standard anatomy. In another example, more of the chamber 904 may be occupied by the fluid permeable body 920 when the intended wearer is a female with standard anatomy or a male with nonstandard anatomy (e.g., micro or buried anatomy).

Fluid collection devices described herein may be used in fluid collection systems. The fluid collection systems can include a fluid collection device, a fluid storage container, and a portable vacuum source. Fluid (e.g., urine or other bodily fluids) collected in the fluid collection device may be removed from the fluid collection device via a conduit which protrudes into an interior region of the fluid collection device. For example, a first open end of the conduit may extend into the fluid collection device to a reservoir therein. The second open end of the conduit may extend into the fluid collection device or the portable vacuum source. The suction force may be introduced into the interior region of the fluid collection device via the first open end of the conduit responsive to a suction (e.g., vacuum) force applied at the second end of the conduit. The suction force may be applied to the second open end of the conduit by the portable vacuum source either directly or indirectly.

In some embodiments, the portable vacuum source may be disposed in or on the fluid collection device. In such embodiments, the conduit may extend from the fluid collection device and attach to the portable vacuum source at a first point therein. An additional conduit may attach to the portable vacuum source at a second point thereon and may extend out of the fluid collection device, and may attach to the fluid storage container. Accordingly, a vacuum (e.g., suction) may be drawn through fluid collection device via the fluid storage container. Fluid, such as urine, may be drained from the fluid collection device using the portable vacuum source.

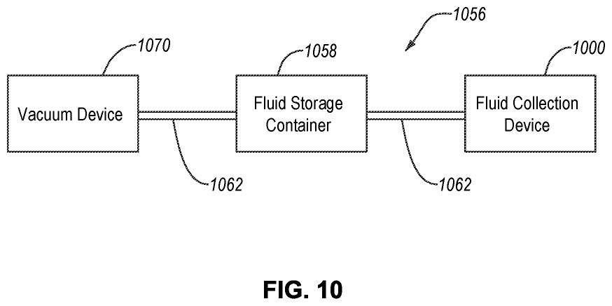

FIG. 10 is a schematic illustration of system 1056 that includes a fluid collection device 1000 according to at least one embodiment of the disclosure. The fluid collection device 1000 may include any of the fluid collection assemblies disclosed herein, such as fluid collection device 100, 500, 600, 700, 800 and/or 900. The fluid collection device 1000 may be in fluid communication with a fluid storage container 1058 via at least one first conduit 1008 (e.g., conduit 108 of FIGS. 1A-B). The fluid storage container 1058 is positioned downstream from the fluid collection device 1000. The fluid storage container 1058 may be in fluid communication with a vacuum device 1070 via at least one second tube 1062. The vacuum device 1070 is positioned downstream from the fluid storage container 1058. During operation, the vacuum device 1070 provides a suction force to the fluid collection device 1000. The suction force draws fluid into the chamber and towards the first conduit 1008. The fluid that enters the first conduit 1008 is pulled by the suction force towards the fluid storage container 1058 such that the fluid storage container 1058 receives the fluid. The fluid storage container 1058 may be configured to inhibit the fluid from flowing from the fluid storage container 1058 to the vacuum device 1070.

In any of the examples, systems or devices disclosed herein, the system of fluid collection device may include moisture sensors (not shown) disposed inside of the chamber of the fluid collection device. In such examples, the moisture sensor may be operably coupled to a controller or directly to the portable vacuum source, and may provide electrical signals indicating that moisture is or is not detected in one or more portions of the chamber. The moisture sensor(s) may provide an indication that moisture is present, and responsive thereto, the controller or portable vacuum device may direct the initiation of suction to the chamber to remove the fluid therefrom. Suitable moisture sensors may include capacitance sensors, volumetric sensors, potential sensors, resistance sensors, frequency domain reflectometry sensors, time domain reflectometry sensors, or any other suitable moisture sensor. In practice, the moisture sensors may detect moisture in the chamber and may provide a signal to the controller or portable vacuum source to activate the portable suction device.

The fluid collection devices and adapters disclosed herein may permit fluid collection devices to accommodate both male and female wearers. The fluid collection devices and adapters disclosed herein may permit fluid collection devices to accommodate wearers with nonstandard anatomy. In some applications, the fluid collection devices and adapters disclosed herein may reduce the need to purchase different devices for different wearers. In some applications, the fluid collection devices and adapters disclosed herein may provide improved performance for wearers with nonstandard anatomy.

As used herein, the term “about” or “substantially” refers to an allowable variance of the term modified by “about” or “substantially” by ±10% or ±5%. Further, the terms “less than,” “or less,” “greater than,” “more than,” or “or more” include, as an endpoint, the value that is modified by the terms “less than,” “or less,” “greater than,” “more than,” or “or more.”

While various aspects and embodiments have been disclosed herein, other aspects and embodiments are contemplated. The various aspects and embodiment disclosed herein are for purposes of illustration and are not intended to be limiting.

We claim:

1. A fluid collection device, comprising:
a fluid impermeable layer having an inner surface at least partially defining a chamber, a first end region defining an aperture extending therethrough and including a first outer surface portion, and a second end region distal to the first end region, the fluid impermeable layer also defining an opening;
a fluid permeable layer positioned at least partially within the chamber to extend across at least a portion of the opening and configured to wick fluid away from the opening; and
a shape memory layer configured to maintain a shape into which the shape memory layer is manipulated into,
wherein the fluid collection device defines at least one substantially planar surface that manipulatable into a plurality of configurations.

2. The fluid collection device of claim 1, wherein the shape memory layer is disposed between the fluid impermeable layer and the fluid permeable layer.

3. The fluid collection device of claim 1, wherein the shape memory layer is disposed on a lower surface of the fluid impermeable layer opposite the chamber.

4. The fluid collection device of claim 1, wherein the shape memory layer includes at least one of continuous layer of shape memory material, a plurality of strips of shape memory material, or a mesh of shape memory material.

5. The fluid collection device of claim 1, wherein the shape memory layer includes at least one of a metal or a polymer.

6. The fluid collection device of claim 1, further comprising an end cap configured to couple to the aperture when the fluid collection device is in a first configuration of the plurality of configurations.

7. The fluid collection device of claim 1, further comprising an end cap configured to enclose at least a portion of the first end region when the fluid collection device is in a second configuration of the plurality of configurations.

8. A fluid collection device, comprising:
a fluid impermeable barrier having an inner surface at least partially defining a chamber, wherein a bottom of the chamber is a substantially planar surface, the fluid impermeable barrier further defining a first end region defining an aperture extending therethrough and including a first outer surface portion, and a second end region distal to the first end region, the fluid impermeable barrier also defining an opening;
a fluid permeable body positioned at least partially within the chamber to extend across at least a portion of the opening and configured to wick fluid away from the opening; and
a retractable hood coupled to at least one of the fluid permeable body or the fluid impermeable membrane and configured to extend over at least a portion of the fluid permeable layer when in use, the retractable hood defining at least a portion of an opening and an interior, wherein the opening and interior are configured to accept at least a portion of a wearer's penis.

9. The fluid collection device of claim 8, wherein the retractable hood is coupled to the fluid impermeable barrier.

10. The fluid collection device of claim 9, wherein the retractable hood is coupled to the fluid impermeable barrier with at least one of an adhesive or a plurality of stitches.

11. The fluid collection device of claim 8, wherein the retractable hood is configured to be folded under the fluid permeable body and above the fluid impermeable barrier when not in use.

12. The fluid collection device of claim 8, wherein the retractable hood is configured to be removed when not in use.

13. The fluid collection device of claim 8, wherein the retractable hood includes a fluid impermeable material.

14. The fluid collection device of claim 8, wherein an opening of the retractable hood includes an elastic material.

15. The fluid collection device of claim 8, wherein the fluid impermeable barrier further defines a pair of wings extending from either side of the fluid permeable body.

16. The fluid collection device of claim 8, wherein the fluid impermeable barrier includes an adhesive on a first surface opposite a surface on which the fluid permeable body is disposed.

17. A fluid collection device, comprising:
a fluid impermeable barrier having a concave inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier, wherein a perimeter of the opening defines an oblong shape; and
a fluid permeable body disposed at least partially within the chamber.

18. The fluid collection device of claim 17, wherein the fluid permeable body comprises a plurality of blocks, wherein individual ones of the plurality of blocks are configured to be selectively removed to accommodate a wearer's anatomy.

19. The fluid collection device of claim 18, wherein the fluid permeable body extends beyond the chamber.

20. The fluid collection device of claim 18, wherein the plurality of blocks comprise an open cell foam.

21. The fluid collection device of claim 20, wherein the open cell foam includes polyurethane.

22. The fluid collection device of claim 18 further comprising a liner disposed at the perimeter of the opening.

23. The fluid collection device of claim 22, wherein a surface of the liner includes an adhesive.

24. The fluid collection device of claim 22, wherein the liner comprises a hydrophobic material.

25. The fluid collection device of claim 22, wherein the liner comprises an open cell foam.

26. The fluid collection device of claim 17, wherein the fluid permeable body comprises a plurality of contours to accommodate a wearer's anatomy.

27. The fluid collection device of claim 26, wherein the plurality of contours includes a protrusion configured to be placed between the wearer's labia.

28. The fluid collection device claim 26, wherein the plurality of contours includes a depression configured to accept at least a portion of a wearer's penis.

29. The fluid collection device of claim 26, wherein a portion of the fluid permeable body extends beyond the chamber.

30. The fluid collection device of claim 26, wherein the fluid permeable body is retained within the chamber by an adhesive.

31. A kit including the fluid collection device of claim 26 and a plurality of permeable bodies, each including a different plurality of contours.

32. A fluid collection device, comprising:
a fluid impermeable barrier having a concave inner surface at least partially defining a chamber, a first end region defining an aperture extending therethrough and including a first outer surface portion, and a second end region distal to the first end region, the fluid impermeable barrier also defining an opening extending longitudinally along the fluid impermeable barrier, wherein a perimeter of the opening defines a teardrop shape, wherein a pointed end of the teardrop shape is at the first end region;
a fluid permeable body positioned within at least a portion of the chamber to extend across at least a portion of the opening and configured to wick fluid away from the opening to a reservoir defined by the fluid permeable body and the fluid impermeable barrier, wherein the reservoir is disposed at the first end region, wherein a portion of the chamber occupied by the fluid permeable body is based, at least in part, on anatomy of a wearer,
wherein the fluid collection device is configured to be worn by the wearer such that the first end region is gravitationally lower than the second end region.

33. The fluid collection device of claim 32, further comprising a conduit extending through the aperture to the reservoir, the conduit fluidly coupled to the reservoir.

34. The fluid collection device of claim 32, wherein the fluid permeable membrane includes a fluid permeable support at least partially covered by a fluid permeable membrane.

35. The fluid collection device of claim 34, wherein the fluid permeable support includes an open cell foam.

36. The fluid collection device of claim 34, wherein the fluid permeable membrane comprises a nonwoven material.

37. The fluid collection device of claim 32, wherein the portion of the chamber occupied by the fluid permeable body is less when the wearer is a male with standard anatomy than when the wearer is a female with standard anatomy or a male with nonstandard anatomy.
